(19) United States
(12) Patent Application Publication
(10) Pub. No.: US 2008/0032324 A1
(43) Pub. Date: Feb. 7, 2008

(54) Apparatus and method for cell migration assays

(75) Inventors: David R. Walt, Boston, MA (US); Christopher J. Di Cesare, Salem, MA (US)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(73) Assignee: The Trustees Of Tufts College

(21) Appl. No.: 11/729,431

(22) Filed: Mar. 28, 2007

Related U.S. Application Data
(63) Continuation of application No. PCT/US05/34627, filed on Sep. 28, 2005.
(60) Provisional application No. 60/614,196, filed on Sep. 28, 2004.

(51) Int. Cl. C12N 1/02; C12M 1/34

(52) U.S. Cl. 435/29; 435/287.1

(57) ABSTRACT

The present invention is directed to a method and apparatus for detecting and analyzing cell migration. More specifically, the present invention is directed to novel technology for analyzing cellular movement, including whole cell migration and subcellular component movement. Cells are distributed onto a substrate and monitored for migration or movement. According to one embodiment, when a labelled cell or portion of a cell passes over one of the delineations between detection units, such as individual fibers in a fiber optic bundle, the label causes a large intensity increase, which stays for a given “residence time” until the cell departs from the detection unit.

### FIELD OF THE INVENTION

The present invention relates to an apparatus and method for detecting cell migration. In addition the invention relates to a method of detecting migration of intracellular organelles in living cells.

### BACKGROUND OF THE INVENTION

Cells from a malignant tumor are able to leave the original site and establish metastases throughout the body through a combination of adhesion, proteolysis, and migration. The importance of studying cell migration becomes apparent when it is realized that the prevention of cellular migration can stop cancer proliferation. Until recently, chemotherapy regimens consisted entirely of antiproliferative compounds, which typically operate by inhibiting nucleic acid function. Antiproliferative drugs are harmful to most dividing cells, causing side effects such as immune suppression, nausea, and kidney damage. The side effects of these compounds have driven the development of alternative strategies to preventing cancer proliferation. One such strategy is using antimigratory compounds, which generally have lower toxicity than antiproliferative chemotherapeutic agents. Antimigratory compounds offer the ability to prevent further metastases by halting cell migration, thereby confining the tumor to its original location.

In addition, cell adhesion and migration are important in a variety of biological processes in addition to tumor progression and metastasis, including development and inflammation, as well as a number of immunology applications.

Several techniques have been used in the past to characterize cell migration and to facilitate the discovery of new antimigratory drugs. Techniques such as the Boyden chamber and the wound assay are among the most widespread techniques used to study cell migration, but suffer from several limitations. The Boyden chamber is a well that contains a floor made of porous material. The pores are generally tailored to be roughly the same size as the cells used in an experiment. Fluorescently dyed cells are allowed to move through the porous material, and are detected once they have moved through to the opposite side. The Boyden chamber relies on a chemical gradient to produce directed cell movement, and cannot be used for examining individual cell migration. In addition, characteristics of the platform itself can cause variations in migration, such as pore size and gradient dissipation. In the wound assay, cells are allowed to grow to confluency. The confluent layer is then cut (or “wounded”), and allowed to resettle on the dish surface. After resettling, the monolayer moves in through the “wound” to recreate the confluent layer. The nature of the wound assay also makes it difficult to examine individual cells, as the entire group is a dynamic monolayer of cells. Although these two methods are very useful, they are population-based techniques that examine a collective response from many cells, and consequently miss important details such as cell-substrate adhesion and intracellular motion.

More recently developed techniques, such as cell patterning and software tracking have the ability to record the movement of individual cells, but these techniques involve complex procedures and/or computer software, and generally take from hours to days to determine drug efficacy.

Accordingly, there is a need in the art for devices and methods for detecting cell migration on a single cell level in a short amount of time.

### BRIEF SUMMARY OF THE INVENTION

The present invention, in one embodiment, is directed to a method of detecting cell migration. The method includes providing a plurality of cells on a substrate having a plurality of detection units, wherein each of the cells is disposed over at least one of the detection units. The method further includes detecting cellular movement of at least one of said cells. In one alternative aspect of the invention, each of the cells is disposed over more than one of the detection units. According to a further alternative, the method also includes adding an analyte of interest prior to the detecting cellular movement. In yet another alternative the method includes adding an analyte of interest after the detecting cellular movement and detecting cellular movement of at least one of the cells a second time after adding said analyte. According to one embodiment, the detecting comprises measuring residence time of intensity of a label within a detection unit.

The present invention, in another embodiment, is directed to a composition for detection of cellular migration. The composition has a substrate and a plurality of cells distributed on the substrate. The substrate has a plurality of detection units. Each of the cells is disposed over at least one of the detection units. In one embodiment, the composition also includes a detection component.

While multiple embodiments are disclosed, still other embodiments of the present invention will become apparent to those skilled in the art from the following detailed description, which shows and describes illustrative embodiments of the invention. As will be realized, the invention is capable of modifications in various obvious aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1. Experimental setup for fiber bundle based cell migration studies. (A) A fiber bundle is inserted through a hole drilled into the bottom of a delta T4 dish to allow for temperature control. Fluorescently dyed cells are deposited on the fibronectin-coated bundle. (B) Schematic of the light source, fiber, and detector, based on an inverted microscope setup. Light emanating from the source is passed through a filter set, guided through the optical fiber, and excites the fluorescent molecules in the cell membrane near the surface of the fiber bundle distal end. The resulting fluorescent emission is then guided back through the individual fibers to the detector.

FIG. 2. Whole-cell migration analysis. (A) Pseudocolor images, taken at the beginning of an experiment, and after ten hours had elapsed. Spots of high (red) and mid-level intensities (green) shifted during this time, indicating cell movement. (B) In this schematic, a fiber begins with background intensity. Yellow represents the fiber core that transmits light and black represents the opaque cladding material. As a dyed cell migrates over the fiber, the intensity increases, and then diminishes as the cell continues to migrate off the fiber. The residence time is calculated by measuring the amount of time the intensity level remained above a set threshold. (C) Raw data taken from a control experiment. Each trace represents an individual fiber. Intensity spikes indicate cell movement over that fiber. For drug-exposed cells, intensity spikes have a longer duration (data not shown). (D) Graph of average residence times of 30 fibers from control, 200 nM, and 300 nM nocodazole experiments. Residence time increases upon nocodazole exposure due to the reduction in cell movement speed.

FIG. 3. Subcellular imaging and analysis. (A) Time-lapse images of subcellular fluorescent intensity changes within two live cells in a control experiment. The pattern of high (red) and mid-intensity (green) fibers changed within 5 minutes from the start of the experiment. (B) The same cells shown in FIG. 3A after exposure to 300 nM nocodazole. The intensity pattern remains relatively constant throughout the 5-minute experiment time. (C) Residence times of cells exposed to various nocodazole concentrations. Each column represents a separate fiber bundle exposed to a different concentration. The increase in residence time indicates a decrease in cell membrane motion.

FIG. 4. A table providing a partial listing of a various types of dyes and their corresponding excitation and emission wavelengths that have utility in the present invention.

FIG. 5. A table providing a partial listing of a various types of dyes and their corresponding excitation and emission wavelengths that have utility in the present invention.

FIG. 6. A table providing a number of indicators which would have particular utility for the biosensor array of the present invention, together with their characteristic excitation and emission wavelengths.

FIG. 7. A table providing a number of indicators which would have particular utility for the biosensor array of the present invention, together with their characteristic excitation and emission wavelengths.

FIG. 8. A table providing a number of indicators which would have particular utility for the biosensor array of the present invention, together with their characteristic excitation and emission wavelengths.

FIG. 9. A table providing a number of indicators which would have particular utility for the biosensor array of the present invention, together with their characteristic excitation and emission wavelengths.

FIG. 10. A table providing a number of indicators which would have particular utility for the biosensor array of the present invention, together with their characteristic excitation and emission wavelengths.

FIG. 11. A table providing a number of indicators which would have particular utility for the biosensor array of the present invention, together with their characteristic excitation and emission wavelengths.

### DETAILED DESCRIPTION

The present invention relates to a method for detecting movement or migration of a cell or organelle within a cell. That is, the method of the present invention, according to one embodiment, relates to detecting cellular movement, where “cellular movement” is intended in the instant application to encompass movement of cells or cellular organelles. In one embodiment, the method includes providing or distributing cells on a substrate that, according to one embodiment, includes a plurality of detection units and detecting cellular movement in relation to at least one detection unit. According to another aspect of the invention in which movement of cellular organelles is detected, cells are distributed on a substrate that includes a plurality of detection units and movement of intracellular organelles is detected by detecting the image of the organelle moving in relation to at least one detection unit.

In a further aspect, the present invention relates to compositions for detecting cell or organelle movement within a cell. The composition comprises a substrate with a plurality of detection units. In one aspect of the invention, the composition is an array. In yet another aspect, the present invention is an apparatus for detecting cellular movement.

According to one embodiment, these methods, compositions, and apparatuses can be used to identify or test cell migration enhancing compounds or antimigratory compounds (compounds that inhibit migration of cells). Alternatively, the applications of the present invention can also include monitoring particle movement, multi-organelle interactions, and cell-substrate events.

Compositions and Apparatuses

In general, the methods of the invention are accomplished by distributing cells on a substrate that includes a variety of discrete detection units. In some embodiments, these cells or cellular organelles are labelled. Thus, as the cells or subcellular components migrate, they travel over different detection units. By monitoring the change in signal from the detection units (e.g., a loss in signal as a cell passes over or leaves on unit and an increase in signal as the cell travels into another), very quick and sensitive methods for cell migration detection are achieved. In the case of cellular organelles or subcellular components, this allows for very rapid analyses.

Accordingly, according to one embodiment, the present invention relates to an apparatus or composition having a substrate with a plurality of detection units. One or more cells can be deposited, distributed, or disposed on the substrate such that each of the cells is disposed on, above, or near a detection unit. In an alternative embodiment, the apparatus or composition can include an optical or detection component for detecting and recording cellular movement on the substrate. In accordance with a further alternative, the apparatus or composition can also include a central processing unit for collection, storage, and processing of the data relating to cellular movement.

According to one embodiment, the present invention relates to a substrate having a plurality of detection units. One or more cells can be deposited, distributed, or disposed on the substrate such that each of the cells is disposed on, above, or near a detection unit.

By “substrate” or “solid support” or other grammatical equivalents herein is meant any material that can be modified to contain discrete units for detecting movement of cells, is appropriate for the attachment or association of cells and is amenable to at least one detection method. As will be appreciated by those in the art, the number of possible substrates is very large. Possible substrates include, but are not limited to, glass and modified or functionalized glass, plastics (including acrylics, polystyrene and copolymers of styrene and other materials, polypropylene, polyethylene, polybutylene, polyurethanes, TeflonJ, etc.), polysaccharides, nylon or nitrocellulose, resins, silica or silica based materials including silicon and modified silicon, carbon, metals, inorganic glasses, plastics, optical fiber bundles, and a variety of other polymers. In general, the substrates allow optical detection and do not themselves appreciably fluorescese.

Generally the substrate is flat (planar), although as will be appreciated by those in the art, other configurations of substrates may be used as well. According to one embodiment, substrates useful in this invention can include optical fiber bundles as discussed below, and flat planar substrates such as glass, polystyrene and other plastics and acrylics.

According to one embodiment, the substrate is a charge-coupled device (“CCD”). As is known in the art, a CCD is a sensor for recording images, consisting of an integrated circuit containing an array of linked, or coupled, capacitors. Typically, CCDs containing grids of pixels are used as light-sensing devices. In the present invention according to one embodiment, the substrate is a CCD in which each of the pixels is a detection unit.

According to one embodiment, the substrate is an optical fiber bundle or array, as is known in the art. In one aspect of the invention in which the substrate is a fiber optic bundle, each fiber or strand is a detection unit. Certain embodiments utilize preformed unitary fiber optic arrays. By “preformed unitary fiber optic array” herein is meant an array of discrete individual fiber optic strands that are co axially disposed and joined along their lengths. The fiber strands are generally individually clad. However, one aspect that distinguishes a preformed unitary array from other fiber optic formats is that the fibers are not individually physically manipulatable. One strand generally cannot be physically separated at any point along its length from another fiber strand. In general, the discussion herein is focused on fiber optic arrays, although as will be appreciated by those in the art, other substrates as described above may be used in any embodiment described herein.

Examples of fiber optic arrays that may be useful in the present invention, are disclosed in U.S. Pat. Nos. 6,327,410, 6,023,540, 6,266,459, 6,859,570, and U.S. application Ser. No. 10/920,637, all of which are incorporated herein by reference.

Optical fiber bundles typically contain hundreds to thousands of hexagonal- or square-packed individual fibers, drawn together so that each fiber carries an isolated signal. The difference in refractive index between the core of each fiber and the surrounding cladding ensures that there is no signal crosstalk between adjacent fibers, allowing a coherent image to pass between each end of the fiber bundle.

In one aspect of the invention, the fiber stands of the present invention range in size from about 4.5 μm to more than 25 μm. Alternatively, the fiber strands can be any known size.

According to one embodiment, the size of the fiber strands used in the present invention can be determined by the measurements to be made. That is, larger fiber strands are used for detection of whole cell movement, while smaller fiber strands are used for subcellular component movement. Alternatively, whole cell movement can be detected using a fiber strand of any known size.

In one embodiment, the surface of the substrate is modified to contain biologically modified sites or is treated with a substance that can be used for attachment of the cells to the substrate.

In one embodiment, the substrate is coated with a thin film of biologically compatible material. Such materials include, but are not limited to, materials known to support cell growth or adhesion may be used, including, but not limited to, fibronectin, any number of known polymers including collagen, polylysine and other polyamino acids, polyethylene glycol and polystyrene, growth factors, hormones, cytokines, etc.

The sensor array design can accommodate a variety of cell sizes and configurations utilizing either commercially available optical fibers and fiber optic arrays or custom made fibers or fiber arrays.

As mentioned above, the present invention according to one embodiment is an array. By “array” herein is meant a plurality of cells in an array format; the size of the array will depend on the composition and end use of the array. Arrays containing from about 2 different cells to many millions can be made, with very large fiber optic arrays being possible. Generally, the array will comprise from two to as many as a billion or more, depending on the size of the cells and the substrate, as well as the end use of the array, thus very high density, high density, moderate density, low density and very low density arrays may be made. Ranges for very high density arrays can be from about 10,000,000 to about 2,000,000,000 (all numbers herein are per cm²), or alternatively from about 100,000,000 to about 1,000,000,000. High density arrays range about 100,000 to about 10,000,000, or from about 1,000,000 to about 5,000,000. Moderate density arrays range from about 10,000 to about 100,000, or alternatively from about 20,000 to about 50,000. Low density arrays are generally less than 10,000, or alternatively from about 1,000 to about 5,000. Very low density arrays are less than 1,000, or from about 10 to about 1000, or alternatively from about 100 to about 500. In some embodiments, the compositions of the invention may not be in array format; that is, for some embodiments, compositions comprising a single cell may be made as well. In addition, in some arrays, multiple substrates may be used, either of different or identical compositions. Thus for example, large arrays may comprise a plurality of smaller substrates.

In addition, one advantage of the present compositions is that particularly through the use of fiber optic technology, extremely high density arrays can be made. Thus for example, because cells frequently may be 200 fm or less and very small fibers are known, it is possible to have as many as 250,000 or more (in some instances 1 million) different fibers and cells in a 1 mm² fiber optic bundle, with densities of greater than 15,000,000 individual cells and fibers (again, in some instances as many as 25-50 million) ner 0.5 cm² obtainable.

By “detection units” is meant a repeating unit on the substrate that allows for independent detection within the unit. According to one embodiment, these units do not comprise physically altered sites. That is, there are no physical barriers between detection units. Instead, the detection units are optically separate repeating units. According to one embodiment, such a configuration is accomplished by a smooth substrate surface allowing for full mobility along the surface by any cells, while the substrate is comprised of a plurality of detection units beneath or adjacent to the surface, wherein each of the detection units is optically separate from each other. This allows for detection of cell or organelle migration without imposing a physical barrier between detection units that would impede cell migration.

The sites may be a pattern, i.e. a regular design or configuration, or randomly distributed. One embodiment utilizes a regular pattern of sites such that the sites may be addressed in the X Y coordinate plane. “Pattern” in this sense includes a repeating unit cell, including one that allows a high density of cells on the substrate.

According to one aspect of the invention, the detection units are smaller than the size of the cell to be examined. That is, upon distribution of the cells on the substrate, the cells reside over more than one detection unit. In one aspect of the invention, the range of number of units “covered” or encompassed by one cell (units per cell) will vary with the assay, with higher ranges preferred for organelle movement. In general, the range of detection units per cell varies from less than about 1 unit/cell to more than about 1,000 units/cell. Alternatively, the range varies from 1 unit/cell to about 100 units/cell. In a further alternative, the range varies from about 2 units/cell to about 50 units/cell.

In one aspect of the present invention, as discussed above, cells are provided or distributed on a substrate. Virtually any cell type and size can be accommodated in the assays of the present invention because the cells are distributed on the substrate irrespective of the size of the detection units. This is because, according to one embodiment, the cells are larger than the size of a detection unit, e.g. each cell occupies or covers more than one detection unit. Virtually any naturally occurring or genetically engineered (i.e. containing exogeneous nucleic acid) eukaryotic or procaryotic cell type may be used, with plants, invertebrates, bacteria and mammalian cells, including, but not limited to, primate, rodent and human cells and cell lines, as well as mixtures of cell types.

In one embodiment, NIH 3T3 mouse fibroblast cells are employed. These cells are typically 15-20 um in size. Other cells types such as E. coli bacteria, 1×3 um, staphylococcus bacteria, approximately 1 um, myoblast precursors to skeletal muscle cells, 15 20 um, neutrophil white blood cells, 10 um, lymphocyte white blood cells, 10 um, erythroblast red blood cells, 5 um, osteoblast bone cells, 15 20 um, chondrocyte cartilage cells, 15 20 um, basophil white blood cells, 10 um, eosinophil white blood cells, 10 um, adipocyte fat cells, 20 um, invertebrate neurons (Helix aspera), 125 um, mammalian neurons, 4 140 um, or adrenomedullary cells, 13 16 um, melanocytes, 20 um, epithelial cells, 20 um, or endothelial cells, 15 20 um, may be utilized as well. Additional other suitable cell types include, but are not limited to, tumor cells of all types (particularly melanoma, myeloid leukemia, carcinomas of the lung, breast, ovaries, colon, kidney, prostate, pancreas and testes), cardiomyocytes, endothelial cells, epithelial cells, lymphocytes (T cell and B cell), mast cells, eosinophils, vascular intimal cells, hepatocytes, leukocytes including mononuclear leukocytes, stem cells such as haemopoetic, neural, skin, lung, kidney, liver and myocyte stem cells, osteoclasts, chondrocytes and other connective tissue cells, keratinocytes, melanocytes, liver cells, kidney cells, and adipocytes. Suitable cells also include known research cells, including, but not limited to, Jurkat T cells, NIH3T3 cells, CHO, COS, etc. A particularly useful source of cell lines may be found in ATCC Cell Lines and Hybridomas (8th ed., 1994), Bacteria and Bacteriophages (19th ed., 1996), Yeast (1995), Mycology and Botany (19th ed., 1996), and Protists: Algae and Protozoa (18th ed., 1993), available from American Type Culture Co. (Rockville, Md.), all of which are herein incorporated by reference.

In accordance with one aspect of the invention, the cells comprise a plurality of cells types. That is, the cells may include a co-culture of cell cultures or cells may be derived from tissue with a mixture of cells. This may be of particular interest in cases where different cell types are known to migrate together, or where migration of one cell type towards another (e.g., in the immune system) is known.

By “cellular organelles” or “subcellular components” is meant any small structures within cells that perform dedicated functions. Cellular organelles include, but are not limited to, the nucleus, mitochondria, Golgi Apparatus, endoplasmic reticulum, lysosomes, endosomes, vacuoles, vesicles, ribosomes, and microtubules.

In a further embodiment, the present invention includes an optical or detection apparatus for detecting and recording cellular movement on the substrate. That is, the present invention, in one embodiment, provides a detection apparatus to optically interrogate cells and monitor cell responses that employs conventional optical components known to one of ordinary skill in the art. Where external optical stimulation of cells is required to elicit an optical response, the detection apparatus can utilize conventional light sources such as arc lamps, photodiodes, or lasers for illumination or excitation light energy. The detection apparatus can monitor cell responses by conventional detector components such as photomultiplier tubes, photodiodes, photoresistors or charge coupled device (CCD) cameras. In one aspect of the invention, the apparatus can include conventional optical train components, such as lenses, filters, beam splitters, dichroics, prisms and mirrors to convey light to and from such light sources and detectors either to discrete substrate sites or through optical fiber strands where imaging of the cells at the distal end is accomplished by viewing or detecting the signal from the proximal end. According to one embodiment, any particular optical apparatus configuration that is employed in optical measurements provides for optically coupling the cells in the array to detectors and light sources. While a particular configuration that was employed in experimental optical measurements is described below, other configurations may also be employed which are functionally equivalent and appropriate suited for a particular measurement requirement.

The detection apparatus, according to one embodiment, is a microscope operably coupled to the substrate. For example, the microscope can be a IX81 Olympus Inverted Microscope™, available from Olympus in Lake Success, N.Y. In some alternative embodiments, the microscope can be converted from a vertical to a horizontal configuration. Alternatively, the detection apparatus can be any known detection apparatus.

The apparatus, according to another alternative embodiment, further includes a central processing unit or a microcomputer programmed to collect and, in some embodiments, to process the data detected by the apparatus. The computer is operably coupled to the apparatus such that the data from the detection component is transferred electronically to the computer.

Methods of Detection of Cellular Movement

In use, according to one embodiment, the above apparatus or composition of the present invention can be used for a method of detecting cell migration that relates to detecting cellular movement of cells distributed on a substrate. According to one embodiment, the method generally includes providing or distributing cells onto the substrate and then detecting cellular movement. The detection step, according to one embodiment, relates to quantifying the amount of time spent by a moving cell or subcellular component at a detection unit. According to one embodiment, the method also includes adding an analyte of interest or candidate agent to examine the interactions between the cells and the analyte or agent. In one alternative aspect of the invention, the cells are labelled prior to detection. The method in accordance with one embodiment includes collection of data relating to cellular movement and can also include processing of that data, including statistical analysis. In another embodiment, prior to distribution, the cells can first be cultured and placed in suspension or otherwise prepared for distribution. The method can also include investigation of cell characteristics such as viability, according to one embodiment.

According to one embodiment of the method, cells to be examined are distributed on the substrate. In one aspect of the invention, the cells are cultured as described below, placed in suspension, and then distributed on the substrate. Alternatively, the cells are grown on the substrate containing the detection units. In a further alternative, the cells are grown on a conventional substrate and a second substrate containing the detection unit is inserted through or integrated with the first substrate for detection of the cells.

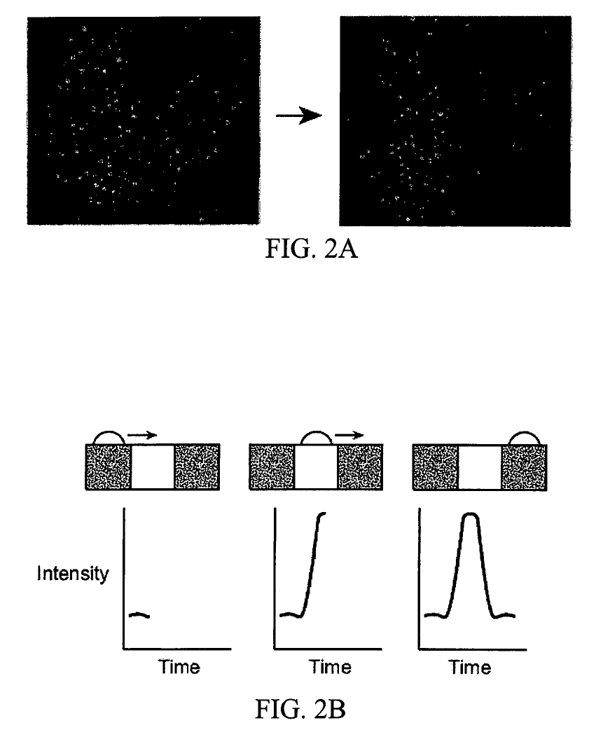

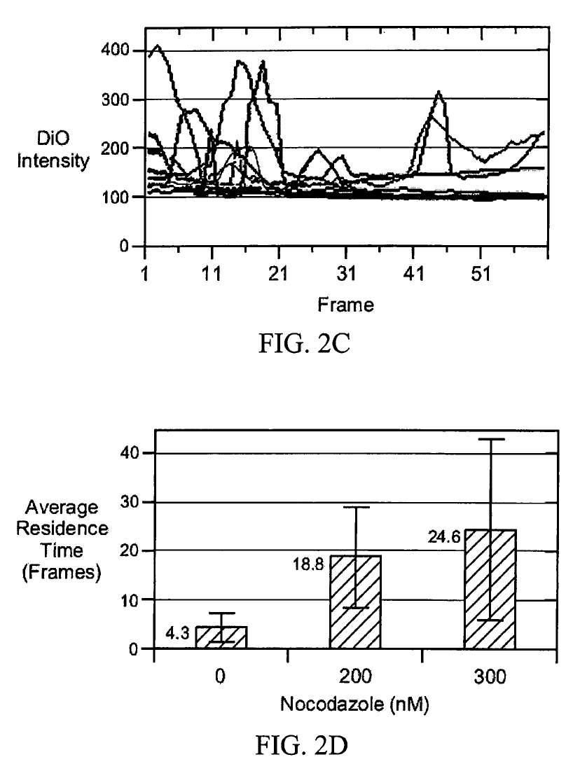

Cellular movement is then examined or detected. The detecting step can occur as a result of movement of a cell or a portion of a cell in relation to a detection unit. That is, because the substrate surface is smooth, cells do not preferentially adhere and localize directly over a detection unit, but will migrate over multiple units during the course of an experiment. Movement does not have to be directed or tracked as with alternative methods, because random movement of cells over the substrate can be quantified by the residence time over every individual detection unit as illustrated by FIG. 2B. Thus, cellular movement is quantified as the amount of time spent by a moving cell at a detection unit (referred to herein as “residence time”).

The present invention relates in one embodiment to the detection of the labeled cell within detection units and measuring the residence time of the cell or cell image in a particular detection unit. An increase or decrease in cellular migration is evidenced by a change in residence time of a cell or organelle, e.g. the image of the cell or organelle. For example, signal intensity increases as the cell or organelle moves into a detection unit and decreases as the cell depicts a detection unit. Also, when screening for cell migration modulators, signal intensity increases in the presence of a cell migration. Likewise, migration of organelles within a cell are analyzed by detecting images of the labeled organelle and measuring the residence time of the label in detection units.

According to one embodiment, the present invention relates to a method of detecting movement of components within a cell. The detecting step in this embodiment occurs as a result of movement of a organelle or subcellular component within a cell in relation to a detection unit. According to one embodiment, a substrate having smaller detection units is used for the detection of subcellular component movement. That is, smaller detection units allow for detection of motion of smaller components such as organelles. In one aspect of the invention, subcellular events can be correlated to whole cell migration such that detection of subcellular movement can indicate whole cell movement.

One advantage of the method of detecting movement of subcellular components, according to one embodiment, is the speed of detection. That is, movement of subcellular components can be detected quickly, resulting in short experimental times and fast results. Due to the short experimental times, multiple experiments can be performed in succession within a few hours. This decrease in experimental time gives the platform a significant advantage over more common techniques known in the art, which typically take hours to perform a single experiment, and should enable high throughput cell migration assays. Further, the short assay times can allow for re-use of the same cells on the same substrate for successive candidate agent exposures.

In one aspect of the invention, the step of detecting cellular movement can include simultaneous detections using the same imaging system. That is, when fiber optic bundles are used as substrates, several bundles can be imaged simultaneously using the same imaging system, thereby further increasing throughput. In one aspect of the invention, simultaneous experiments can be run by packing multiple fiber bundles together in conjunction with a microtiter plate. That is, the fibers used in this embodiment are long, flexible fibers, and the free distal ends of each fiber bundle are inserted into separate wells of the plate. According to one embodiment, the proximal end of the fibers are all fused together such that all the fiber bundles are brought together so that several bundles can be monitored simultaneously, while exposing them to different conditions on their distal ends. This technique can potentially reduce experimental time by running several fiber-based experiments simultaneously. The potential capacity of this type of experiment may only be limited by the number of fibers that can be fit into the microscope's field of view or by the area of the detection component. This parallel approach may enable scores of rapid, subcellular imaging experiments to be performed concurrently. In addition to their use in running parallel experiments, the use of long, flexible fibers demonstrates remote sensing, as hundreds of centimeters separate the assay and imaging locations.

As mentioned above, according to one embodiment, the present invention provides methods for detecting the responses of individual cells to analytes of interest. Generally, in one embodiment, a candidate bioactive agent is added to the cells prior to analysis, and the cells allowed to incubate for some period of time. By “administration” or “contacting” herein is meant that the candidate agent is added to the cells in such a manner as to allow the agent to act upon the cell, whether by uptake and intracellular action, or by action at the cell surface. In some embodiments, nucleic acid encoding a proteinaceous candidate agent (i.e. a peptide) may be put into a viral construct such as a retroviral construct and added to the cell, such that expression of the peptide agent is accomplished; see PCT US97/01019, hereby expressly incorporated by reference.

Once the candidate agent has been administered to the cells, according to one embodiment, the cells can be washed if desired and are allowed to incubate under preferably physiological conditions for some period of time.

In one embodiment, cells or images thereof are visualized or detected prior to administration of a candidate agent in order to obtain a baseline of motility. Then the candidate agent(s) are added to the cells. Cells are then visualized or detected to determine the effect of the candidate agent. Alternatively, a control culture without a candidate agent is monitored while an experimental culture is contacted with a candidate agent. Again, cells are visualized or detected to determine the effect of the candidate agent.

By “analyte of interest” or “target analyte” or “candidate bioactive agent” or “candidate drug” or grammatical equivalents herein is meant any molecule, e.g., protein, oligopeptide, antibody, small organic molecule. polysaccharide, polynucleotide, etc., to be tested for the ability to directly or indirectly altering cellular motility. Generally a plurality of assay mixtures are run in parallel with different agent concentrations to obtain a differential response to the various concentrations. Typically, one of these concentrations serves as a negative control, i.e., at zero concentration or below the level of detection.

Analytes encompass numerous chemical classes, though typically they are organic molecules, including small organic compounds having a molecular weight of more than 100 and less than about 2,500 daltons. Analytes comprise functional groups necessary for structural interaction with proteins, particularly hydrogen bonding, and typically include at least an amine, carbonyl, hydroxyl or carboxyl group, or alternatively at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids. steroids, purines, pyrimidines, derivatives, structural analogs or combinations thereof. Alternatively, the agents are peptides.

Candidate agents are obtained from a wide variety of sources including libraries of synthetic or natural compounds. For example, numerous means are available for random and directed synthesis of a wide variety of organic compounds and biomolecules, including expression of randomized oligonucleotides. Alternatively, libraries of natural compounds in the form of bacterial, fungal, plant and animal extracts are available or readily produced. Additionally, natural or synthetically produced libraries and compounds are readily modified through conventional chemical, physical and biochemical means. Known pharmacological agents may be subjected to directed or random chemical modifications, such as acylation, alkylation, esterification, amidification to produce structural analogs.

In some embodiments candidate agents are unknown agents and are screened for their ability to modify cell or organelle motility. In other embodiments candidate agents are previously known agents, such as growth factors, angiogenic factors, anti-angiogenic factors, cytokines, and the like.

In one embodiment, the candidate bioactive agents are naturally occurring proteins or fragments of naturally occurring proteins. Thus, for example, cellular extracts containing proteins, or random or directed digests of proteinaceous cellular extracts, may be used. In this way libraries of procaryotic and eucaryotic proteins may be made for screening in the methods of the invention. According to one aspect of the invention, the libraries are libraries of bacterial, fungal, viral, and mammalian proteins, including human proteins.

The peptides may be digests of naturally occurring proteins as is outlined above, random peptides, or “biased” random peptides. By “randomized” or grammatical equivalents herein is meant that each nucleic acid and peptide consists of essentially random nucleotides and amino acids, respectively. Since generally these random peptides (or nucleic acids, discussed below) are chemically synthesized, they may incorporate any nucleotide or amino acid at any position. The synthetic process can be designed to generate randomized proteins or nucleic acids, to allow the formation of all or most of the possible combinations over the length of the sequence, thus forming a library of randomized candidate bioactive proteinaceous agents.

In one embodiment, the library is fully randomized, with no sequence preferences or constants at any position. In one embodiment, the library is biased. That is, some positions within the sequence are either held constant, or are selected from a limited number of possibilities. For example, according to one embodiment, the nucleotides or amino acid residues are randomized within a defined class, for example, of hydrophobic amino acids, hydrophilic residues, sterically biased (either small or large) residues, towards the creation of nucleic acid binding domains, the creation of cysteines, for cross linking, prolines for SH3 domains, serines, threonines, tyrosines or histidines for phosphorylation sites, etc., or to purines, etc.

As described above generally for proteins, nucleic acid candidate bioactive agents may be naturally occurring nucleic acids, random nucleic acids, or “biased” random nucleic acids. For example, digests of procaryotic or eucaryotic genomes may be used as is outlined above for proteins. “Nucleic acids” in this context includes both DNA and RNA, and nucleic acid analogs including PNA. In one aspect of the invention, the candidate bioactive agents are organic chemical moieties, a wide variety of which are available in the literature.

In one embodiment of the invention, the cells to be examined for cellular movement are labelled. The method, according to one aspect of the invention, of detecting the movement of labelled cells includes illuminating the cells with excitation light or energy and detecting the intensity changes in the detection units. That is, movement is quantified as an increase in intensity at a particular detection unit, as a fluorescently-dyed cell moves across that unit.

According to one embodiment in which the cells are labelled with fluorescent labels and the detection units are optical fibers, excitation light is filtered and passed through the fiber bundle, interacting with fluorescent molecules bound to the membranes of cells near the distal face of the fiber (the surface of the substrate). The emission signal is passed back through the fiber, a microscope objective, and emission filter and then detected by a detection device such as a camera. According to one embodiment, data is then collected based on these results, as described in further detail below.

By “labelled” herein is meant that the compound is either directly or indirectly labeled with a label which provides a detectable signal, e.g. radioisotope, fluorescers, enzyme, antibodies, particles such as magnetic particles, chemiluminescers, or specific binding molecules, etc. Specific binding molecules include pairs, such as biotin and streptavidin, digoxin and antidigoxin etc.

Labelling facilitates or enables visual detection of the cells as they move across the substrate between detection units. In one embodiment, the label is a label specific for the membrane. Alternatively, when organelles are to be visualized, a label is provided that can cross the cell membrane and label intracellular membranes. Alternatively, genetically engineered cells expressing fluorescent proteins such as Green Fluorescent Protein or variants thereof as are known in the art are used, and the migration of the expressed protein is detected in the cell. In certain embodiments, the cells comprise a plurality of labels. In some embodiments certain labels serve as markers for the cell or organelle while others serve as indicators of physiological states of the cell, e.g. for detection of pH, or apoptosis (using nexin dyes, for example).

A wide variety of fluorophores, chromophores, stains or a dye compounds may be used for labeling cells. In one embodiment, in selecting the correct dye for determination of cell migration, several features should be considered. Dyes should be relatively intense, photostable, well retained and should not interfere with cell migration. Such interference could be due to a general toxicity, but could also be more specific, such as the inhibition of DNA replication. The encoding dye in accordance with one embodiment is the lipophilic carbocyanine dye Vybrant DiO. DiO, due to its lipophilic nature, can rapidly incorporate into the plasma membrane of a cell and become internalized into membranous organelles. Internal cellular structures are easily dyed with DiO.

Other dyes may be permeant or impermeant to the cell membrane. Impermeant dyes may be conjugated with acetoxymethyl ester to allow take up by cells. In one embodiment, conventional conjugate or reactive cell membrane stains, cell tracers, or cell probes such as fluoresceins, rhodamines, eosins naphthalimides, phycobiliproteins, nitrobenzoxadiazole may be utilized. In other embodiments, cyanine dyes, such as SYTO® (Molecular Probes), amine reactive dyes, thiol reactive dyes, lipopilic dyes, and DNA intercalators, such as acridine orange, may be employed. In one embodiment, fluorogenic or chromogenic enzyme substrates may be taken up by the cells, processed by intracellular enzymes, such as glycosidases, phosphatases, luciferase, or chloramphenicol acetyltransferase, and provide a label.

In an alternative embodiment, cell organelle dye probes may be employed. In one embodiment, cell membrane probes such as carbocyanines and lipophilicaminostyrls may be utilized.

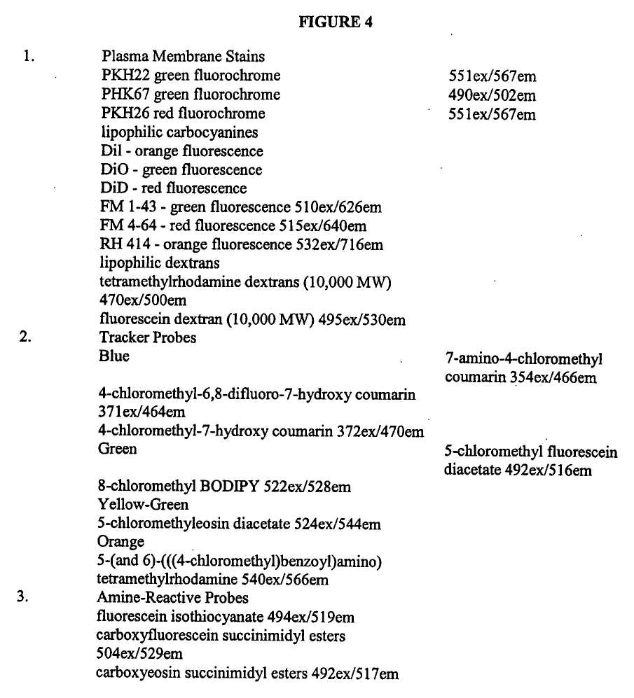

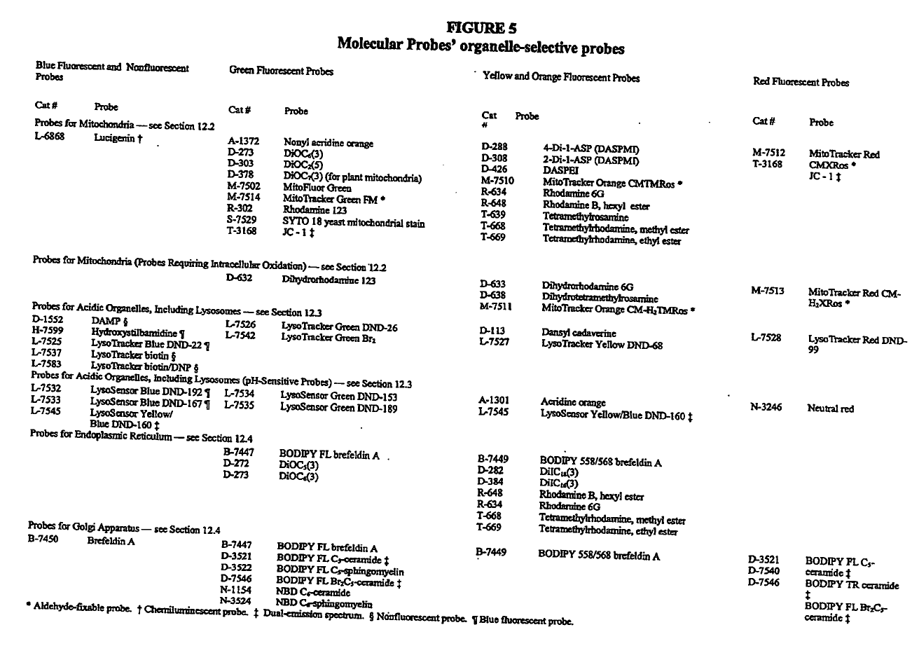

By way of example, FIGS. 4 and 5 depict tables providing a partial listing of a various types of dyes and their corresponding excitation and emission wavelengths that have utility in the present invention. In addition, a particularly useful reference for selecting other types of encoding dyes is R. P. Haugland, Handbook of Fluorescent Probes and Research Chemicals (6th ed.), Molecular Probes Inc. catalog (Eugene, Oreg., 1996), both of which are expressly incorporated herein by reference.

While a number of dyes and methods are available, in one embodiment, external fluorescent cell membrane labels, PKH67 with an excitation wavelength of 490 nm and an emission wavelength of 502 nm, and PKH26 with an excitation wavelength of 551 nm and an emission wavelength of 567 nm, were utilized. PKH26 and PKH67 are part of a family of dyes manufactured by Zynaxis Cell Science (Malvern, Pa.), sold under the trademark Zyn Linker® (Phanos Technologies Inc.), produced by the method of U.S. Pat. No. 5,665,328 to Hogan, et al., and available from Sigma (St. Louis, Mo.).

Alternatively, the cells are not labelled. That is, according to one embodiment, the method of detecting cellular movement relates to detecting movement of unlabelled cells. In one aspect of the invention, the detection can occur by any known method of gauging cell movement. For example, one method could include simple illumination of the cells and optical detection of cell movement. One method of optical detection is a light scattering assay. That is, cellular movement can be detected by measurement of their forward and 90 degree (side) light scatter properties. These scatter properties include the size and shape of the cells and could be used to detect changes in position.

In one aspect of the invention, the method includes collecting the detected data. That is, when a cell or subcellular component moves into proximity with or away from a detection unit, the information relating to the detected movement is collected. For example, in the case of labelled cells, the increased intensity of the emission signal created by the residence time of a cell or subcellular component in relation to a detection component is detected and collected. According to one embodiment, the collection step involves transferring the data to a computer. The detection and collection occurs for each detection unit on a substrate.

Alternatively, the collection step can be limited by certain parameters. For example, according to one embodiment, collection of data can be limited to data from those detection units detecting a minimum emission intensity or residence time. Alternatively, the collection could be limited in other ways. For example, according to one embodiment, only data relating to those labelled cells that create the strongest or most intense emission signals is considered. For example, according to one embodiment, the only data considered relates to those detection units exhibiting 75% of the maximum peak intensity for that unit or higher. One reason for establishing such a threshold is because a relatively high threshold is necessary to ensure that the intensity will drop below the threshold as the signal diminishes. In one embodiment, the number of frames that the intensity stays above the established threshold is counted for each detection unit, and then averaged for all detection units, giving an average residence time.

In another embodiment of the present invention, the method includes processing the collected data. In other words, once the assay is run, the data is analyzed to determine the experimental outcome, i.e. either the presence or absence of a target analyte, the effect of a candidate agent on a cellular phenotype, etc. This is generally done using a computer. Processing, in one embodiment, means any manipulation of the data to better understand the results of cellular movement experiments. For example, the processing can include statistical analysis. That is, the optical signal from each detection unit can be combined and any number of statistical analyses run, as outlined below. This can be done for a variety of reasons. For example, in time varying measurements, redundancy can significantly reduce the noise in the system. For non-time based measurements, redundancy can significantly increase the confidence of the data.

Once obtained, the optical response signals from a plurality of sensor beads within each bead subpopulation can be manipulated and analyzed in a wide variety of ways, including baseline adjustment, averaging, standard deviation analysis, distribution and cluster analysis, confidence interval analysis, mean testing, etc.

In one embodiment, the first manipulation of the optical response signals is an optional baseline adjustment. In a typical procedure, the standardized optical responses are adjusted to start at a value of 0.0 by subtracting the integer 1.0 from all data points. Doing this allows the baseline-loop data to remain at zero even when summed together and the random response signal noise is canceled out. When the sample is a vapor, the vapor pulse-loop temporal region, however, frequently exhibits a characteristic change in response, either positive, negative or neutral, prior to the vapor pulse and often requires a baseline adjustment to overcome noise associated with drift in the first few data points due to charge buildup in the CCD camera. If no drift is present, typically the baseline from the first data point for each bead sensor is subtracted from all the response data for the same bead. If drift is observed, the average baseline from the first ten data points for each bead sensor is substracted from the all the response data for the same bead. By applying this baseline adjustment, when multiple bead responses are added together they can be amplified while the baseline remains at zero. Since all beads respond at the same time to the sample (e.g. the vapor pulse), they all see the pulse at the exact same time and there is no registering or adjusting needed for overlaying their responses. In addition, other types of baseline adjustment may be done, depending on the requirements and output of the system used.

Once the baseline has been adjusted, a number of possible statistical analyses may be run to generate known statistical parameters. Analyses based on redundancy are known and generally described in texts such as Freund and Walpole, Mathematical Statistics, Prentice Hall, Inc. New Jersey, 1980, hereby incorporated by reference in its entirety.

According to one embodiment, signal summing is done by simply adding the intensity values of all responses at each time point, generating a new temporal response comprised of the sum of all bead responses. These values can be baseline-adjusted or raw. As for all the analyses described herein, signal summing can be performed in real time or during post-data acquisition data reduction and analysis. In one embodiment, signal summing is performed with a commercial spreadsheet program (Excel, Microsoft, Redmond, Wash.) after optical response data is collected.

In one aspect of the invention, cumulative response data is generated by simply adding all data points in successive time intervals. This final column, comprised of the sum of all data points at a particular time interval, may then be compared or plotted with the individual bead responses to determine the extent of signal enhancement or improved signal-to-noise ratios as shown in FIGS. 14 and 15.

In one embodiment, the mean of the subpopulation (i.e. the plurality of identical beads) is determined, using the well known Equation 1:

$$μ = \sum \frac{{x}_{i}}{n} \qquad Equation 1$$

In some embodiments, the subpopulation may be redefined to exclude some beads if necessary (for example for obvious outliers, as discussed below).

According to one embodiment, the standard deviation of the subpopulation can be determined, generally using Equation 2 (for the entire subpopulation) and Equation 3 (for less than the entire subpopulation):

$$σ = \sqrt{\frac{\sum {( {x}_{i} - μ )}^{2}}{n}} \qquad Equation 2$$

$$s = \sqrt{\frac{\sum {( {x}_{i} - \overline{x} )}^{2}}{n - 1}} \qquad Equation 3$$

s for the mean, the subpopulation may be redefined to exclude some beads if necessary (for example for obvious outliers, as discussed below).

In one embodiment, statistical analyses are done to evaluate whether a particular data point has statistical validity within a subpopulation by using techniques including, but not limited to, t distribution and cluster analysis. This may be done to statistically discard outliers that may otherwise skew the result and increase the signal-to-noise ratio of any particular experiment. This may be done using Equation 4:

$$t = \frac{\overline{x} - μ}{s / \sqrt{n}} \qquad Equation 4$$

In accordance with one aspect of the invention, the quality of the data is evaluated using confidence intervals, as is known in the art. Confidence intervals can be used to facilitate more comprehensive data processing to measure the statistical validity of a result.

In one embodiment, statistical parameters of a subpopulation of beads are used to do hypothesis testing. One application is tests concerning means, also called mean testing. In this application, statistical evaluation is done to determine whether two subpopulations are different. For example, one sample could be compared with another sample for each subpopulation within an array to determine if the variation is statistically significant.

In addition, mean testing can also be used to differentiate two different assays that share the same code. If the two assays give results that are statistically distinct from each other, then the subpopulations that share a common code can be distinguished from each other on the basis of the assay and the mean test, shown below in Equation 5:

$$z = \frac{{\overline{x}}_{1} - {\overline{x}}_{2}}{\sqrt{\frac{{σ}_{1}^{2}}{{n}_{1}} + \frac{{σ}_{2}^{2}}{{n}_{2}}}} \qquad Equation 5$$

Furthermore, analyzing the distribution of individual members of a subpopulation of sensor elements may be done. For example, a subpopulation distribution can be evaluated to determine whether the distribution is binomial, Poisson, hypergeometric, etc.

In a further alternative, the statistical analysis performed can include clustering algorithms known in the art.

As discussed above, the method of the present invention according to one embodiment can include preparation of the cells prior to distribution. In one aspect of the invention, the cells are prepared by conventional culturing. That is, cell populations are conventionally cultured with growth media which matches cell needs. Culture media is formulated according to either recipes provided by cell line providers, journal articles or reference texts. A particularly useful reference for media preparation is ATCC Quality Control Methods for Cell Lines (2nd ed.), American Type Culture Co. (Rockville, Md.) which is herein incorporated by reference. Alternatively, cells can be cultured by any known means. In a further alternative, cell culturing is not required.

In a further embodiment, previously cultured cells are prepared for distribution on a substrate of the present invention by trypsinizing the cells using aseptic techniques to remove them from the cell culture dish and suspend them in growth media. Alternatively, the cells can be prepared by any known method.

In one embodiment, the cell viability is investigated. According to one embodiment, cell viability is examined in order to help establish that any detected cellular movement is caused by live cells and not caused by other factors acting on dead cells. Various dyes and indicators find use in this aspect of the invention. 2′ 7′ bis(2-carboxyethyl) 5 (and 6) carboxyfluorescein (BCECF AM) which has an excitation wavelength of 505 nm and an emission wavelength of 535 nm and is available from Molecular Probes (Eugene, Oreg.) can be used. In another embodiment a commercial cell viability assay, LIVE/DEAD® from Molecular Probes (Eugene, Oreg.), is employed.

In addition to cell viability, further characteristics of cells can be examined according to another embodiment of the present invention. That is, the optical responses of individual cells and cell populations to chemical or biological stimuli are typically interrogated and detected by coupling individual cells with appropriate indicators which may be either fluorophores, chromophores, stains or a dye compounds. Any suitable indicator or combinations of indicators may be utilized provided the indicator does not compromise cell response.

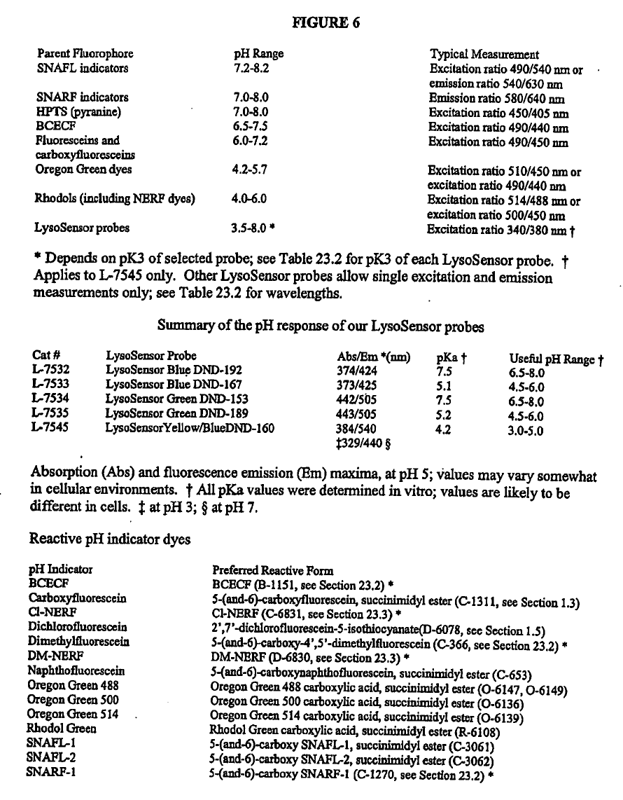

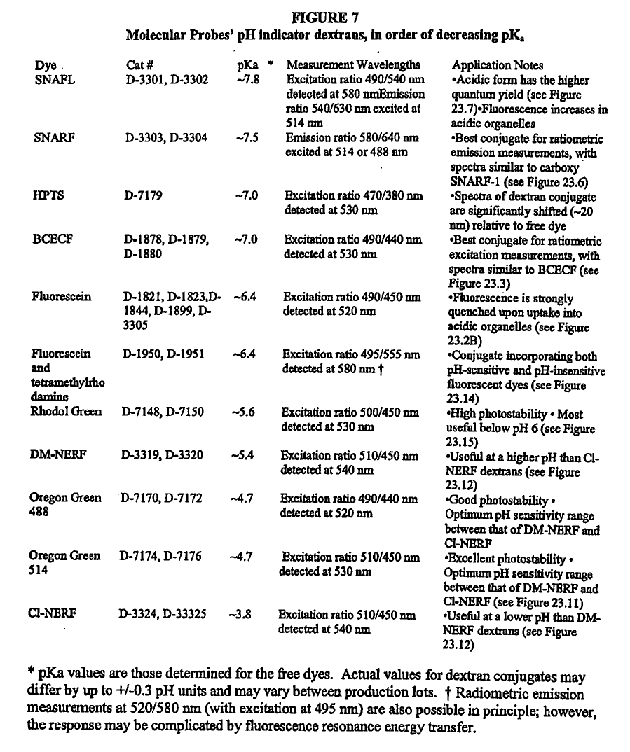

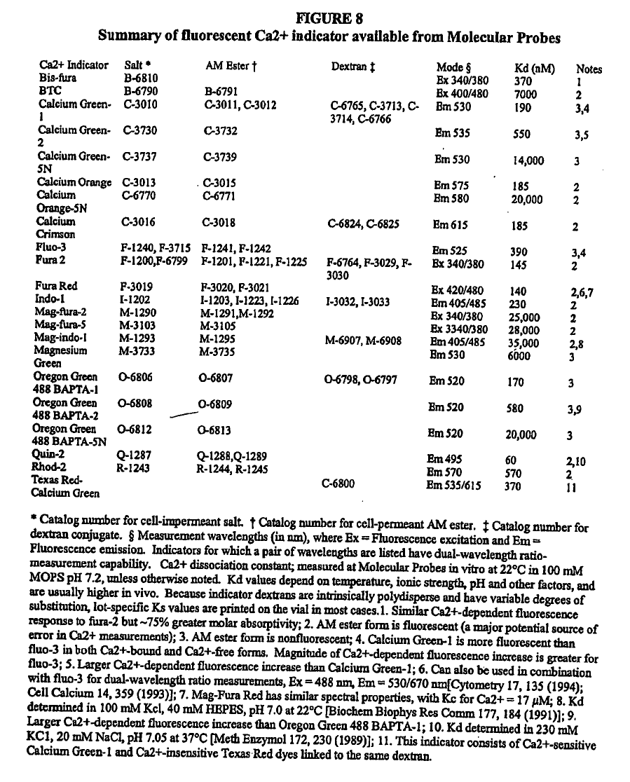

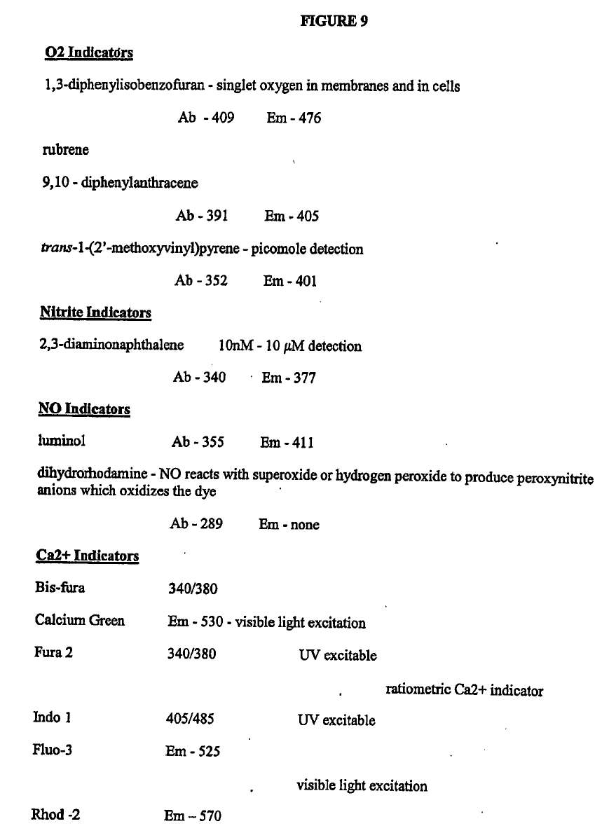

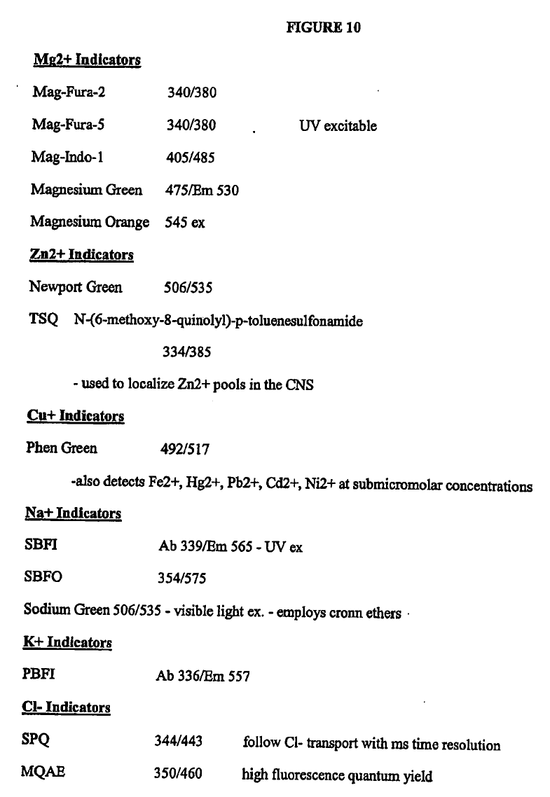

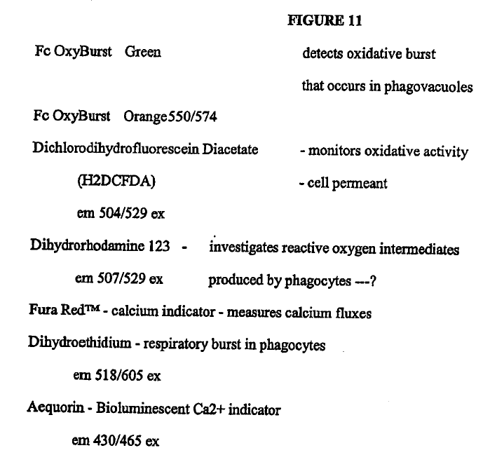

For example, conventional cell fluorophore probes such as fluoresceins, rhodamines, naphthalimides, phycobiliproteins. nitrobenzoxediazole may be utilized. Alternatively, permeant or impermeant cell membrane potential indicators, ion indicators, reactive oxygen indicators and pH indicators may be employed. By way of example a number of indicators which would have particular utility for the biosensor array of the present invention are listed in tables depicted in FIGS. 6 through 11 together with their characteristic excitation and emission wavelengths. A particularly useful reference for selecting appropriate indicators is R. P. Haugland, Handbook of Fluorescent Probes and Research Chemicals (6th ed.), Molecular Probes Inc. (Eugene, Oreg. 1996), which is expressly incorporated herein by reference.

In one embodiment, indicators may be incorporated directly into the cell by attachment to the cell membrane, by absorption into the cell cytoplasm by membrane permeant indicators, or by microinjection into the cell cytoplasm. In another environment, indicators are added to the culture media fluid contained on the substrate.

In one aspect of the invention, the cell migration apparatus and method of the present invention can be applied to a large variety of conventional assays for screening and detection purposes. The apparatus may be configured for virtually any assay and offers a distinct advantage for high throughput screening where a plurality of cells or cell populations, which have utility in particular assays or are genetically engineered cells to provide unique responses to analytes, may be employed in a single sensor array for conducting a large number of cell migration assays simultaneously on a small sample. The biosensor array thus provides both for tremendous efficiencies in screening large combinatorial libraries for agents that affect cell or organelle motility or migration and allows conduction of a large number of assays on extremely small sample volumes, such as biologically important molecules synthesized on micron sized beads.

The biosensor array and method of the present invention utilizes the unique ability of living cell populations to respond to biologically significant compounds in a characteristic and detectable manner, such as motility or migration. Since the selectivity of living cells for such compounds has considerable value and utility in drug screening and analysis of complex biological fluids, a biosensor and assay that makes use of the unique characteristics of living cell populations offers distinct advantages in high throughput screening of combinatorial libraries where hundreds of thousands of candidate pharmaceutical compounds must be evaluated. In addition, such a biosensor and sensing method can be utilized for either off line monitoring of bioprocesses or in situ monitoring of environmental pollutants where the enhanced sensitivity of living cells to their local environment can be exploited.

Another advantage of this technology is the potential to reduce the number of cells and amount of drug used for each migration assay. According to one embodiment, the apparatus of the present invention uses a substrate that closely matches the fiber diameter, minimizing the amount of drug and cells used in an experiment. This reduction in size may allow the use of a cell line that is difficult to grow and maintain, including primary cells. Individual fiber or detection unit sizes can be further decreased, augmenting the range of applications for subcellular imaging.

An additional advantage in the present system is that a plurality of individual cells can be monitored, giving a statistically better analysis.

While the examples below provide a variety of specific assays which may be useful in configuring and employing the biosensor array and method of the present invention, they are not intended to limit either the scope of applications envisioned or the broad range of sensing methods which can be employed with a plurality of cell populations with the biosensor of the present invention.

The reactions outlined herein may be accomplished in a variety of ways, as will be appreciated by those in the art. Components of the reaction may be added simultaneously, or sequentially, in any order. In addition, the reaction may include a variety of other reagents may be included in the assays. These include reagents like salts, buffers, neutral proteins, e.g. albumin, detergents, etc which may be used to facilitate optimal detection, and/or reduce non specific or background interactions. Also reagents that otherwise improve the efficiency of the assay, such as protease inhibitors, nuclease inhibitors, antimicrobial agents, etc., may be used.

While this invention has been particularly shown and described with references to various embodiments thereof, it will be understood by those skilled in the art that various changes in form and details may be made therein without departing from the spirit and scope of the invention as defined by the appended claims.

### EXAMPLES

### Methods

NIH/3T3 fibroblasts were obtained from American Type Tissue Collection (ATCC No. CRL-1658). Dulbecco's modified eagle medium, penicillin-streptomycin, L-glutamine, calf serum, trypsin-EDTA were obtained from Invitrogen (Carlsbad, Calif.). Fibronectin was obtained from Calbiochem-Novabiochem Corporation (La Jolla, Calif.). Live/Dead Viability/Cytotoxicity kit for animal cells and Vybrant DiO cell-labeling solution were obtained from Molecular Probes (Eugene, Oreg.). Nocodazole and sodium bicarbonate were obtained from Sigma Chemical Co. (St. Louis, Mo.). Fiber-optic imaging bundles were obtained from the Schott Corporation (Southbridge, Mass.) and Illumina, Inc. (San Diego, Calif.). Delta T4 culture dish system, and delta T culture dishes were obtained from Bioptechs (Butler, Pa.). Aluminum oxide lapping films were obtained from Mark V Laboratory (East Granby, Conn.).

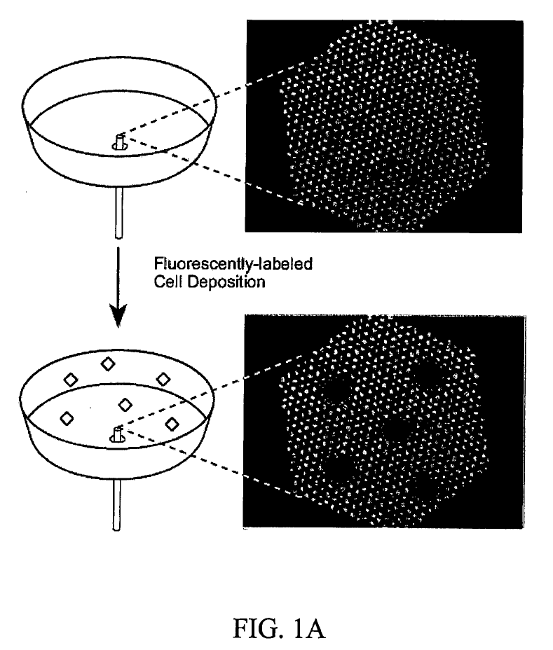

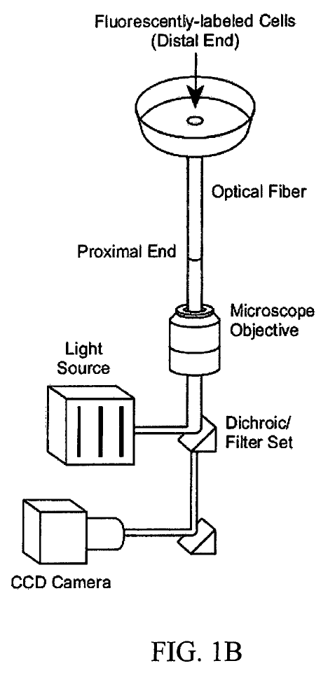

After labeling, the cells were allowed to settle and adhere to a fibronectin-coated fiber bundle that had been secured to a delta T4 dish (FIG. 1A). Fibers were placed into the delta T4 dish to maintain the temperature at 37° C. during experiments. Cells do not necessarily adhere and align directly on top of individual fibers, but may migrate across and span several fibers. After cells were allowed to adhere to the bundle's distal end, it was mounted on an inverted microscope stage, and excitation light was introduced through the proximal end of the fiber bundle. The fiber bundle is not connected directly to any part of the microscope, but remains within a centimeter of the objective. The excitation light was transferred through the fiber bundle to the distal end, where it excited the Vibrant DiO dye in the cell membrane (FIG. 1B). The resulting fluorescence emission light was then carried back through the fiber bundle to a CCD camera. Using this method, the individual cells are not observed; only the fluorescent signal for each cell is obtained.

Cell migration was detected using two types of fiber bundles differing in the sizes of the individual fibers. Larger individual fibers (25± μm) were used to observe whole-cell movement, whereas smaller individual fibers (4.5 μm) were used to image subcellular events.

Fiber bundles measuring 1 mm outer diameter with 25 μm diameter individual fibers (Schott Corporation, Southbridge, Mass.), were used for whole-cell observation. A series of exposures to different nocodazole concentrations were performed, consisting of a control, 200 nM nocodazole, and 300 nM nocodazole, each done on successive days. Images were taken every ten minutes for ten hours. FIG. 2A shows cell migration on a fiber bundle face. All images and movies are shown in pseudocolor to more easily express intensity changes. Individual fibers that are colored red have the highest intensity, green are mid-range, and blue are low intensity (background). In FIG. 2A there are two images of the same fiber, taken at the beginning of an experiment, and ten hours later. It can be seen that the pattern of red fibers (high intensity) at the beginning of the experiment does not match the pattern after ten hours, indicating that cell movement has occurred during the course of the experiment.

Migration is quantified as an increase in intensity at a particular fiber, as a fluorescently dyed cell moves across that fiber. Analysis of confluent layers is most likely less accurate using this technique, as individual cell movement is then restricted. FIG. 2C shows a time series of many individual fiber traces acquired in a control experiment in which no antimigratory drug was used. Each trace is the intensity of an individual fiber over ten hours. Intensity spikes can be clearly seen, indicating when a cell moved over a surface of these particular fibers during the experiment. Larger spikes correspond to red-colored (high intensity) fibers seen in FIG. 2A, and shorter spikes correspond to green-colored fibers (mid-range intensity). All of the fibers in a bundle are examined, and only the fibers with the 30 highest peak intensities were chosen for the final analysis, to ensure that each fiber analyzed had a cell pass over it at some point during the experiment.

In this example, a threshold was assigned for each selected fiber at 75% of the maximum peak intensity for that particular fiber. The amount of time spent above this threshold was considered to be the “residence time” of a cell on that fiber. A relatively high threshold is necessary to ensure that the intensity will cross the threshold a second time as the signal diminishes. The number of frames that the intensity stays above this threshold is counted for each fiber, and then averaged for all 30 fibers, giving an average residence time for the experiment (FIG. 2D). Using this analysis, we determined that all 30 fibers had a cell pass over them at some point during the experiment, so the cell density on the bundle surface does not affect the measurement. The average residence time for the control fiber was 4.3±2.9 frames (ten minutes per frame). When nocodazole is applied, the cell velocity decreases, causing each cell to spend more time over an individual fiber, resulting in a longer residence time for the drug-exposed cells. Exposure to 200 nM resulted in a residence time of 18.8±10.3 frames, and the 300 nM exposure resulted in 24.6±18.6 frames. The high amount of variation from these averages is to be expected as cells vary significantly in their individual responses.

After each experiment, the cells were treated with an ethidium homodimer-1 assay to demonstrate that the cells were alive, and that the responses were due to migration rather than clumping or Brownian motion. These experiments demonstrated that all cells were alive, even after nocodazole treatment.

For subcellular imaging experiments, fiber bundles measuring 1 mm in total diameter, with individual fibers measuring 4.5 μm in diameter were obtained from Illumina, Inc. (San-Diego, Calif.). It was found that performing the migration assay with these smaller fibers gives a significant advantage; experimental time could be reduced from 10 hours to 5 minutes, by measuring subcellular events that can be correlated to whole-cell migration potential.

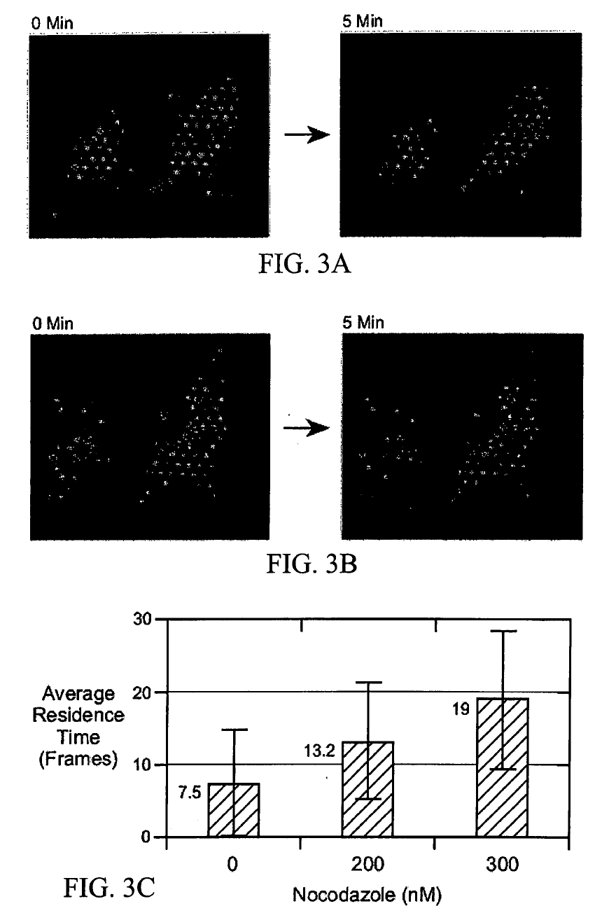

Individual fibers that are covered by cells can be easily selected for analysis, because cell shape is visible through the bundle (FIG. 3A). In the course of a 5-minute control experiment, the pattern of fibers with high and mid-range intensities (red and green) changes significantly (FIG. 3A). Because the assay time is so short, the same cells on the same fiber array could be used for successive drug exposures. The results are shown in FIG. 3B following nocodazole exposure. Intensity changes were observed to be less dynamic upon the addition of nocodazole, as the pattern of high and mid-range intensity fibers changes less during the 5-minute time segment (FIG. 3B). This reduced intensity pattern change corresponds to an increase in the average residence time for the cells on these individual fibers. The subcellular signals are due to internal membranous organelles that have been stained with DiO, as demonstrated with the structurally similar dye DiOCl6. A change in signal is due to movement of these structures within the cells.

The data shown in FIG. 3C originate from an experiment in which three separate fibers were exposed to successively higher concentrations of nocodazole. Analysis is similar to that described for whole cell migration, with a slight modification: only individual fibers that were initially covered by a cell are taken into consideration, rather than examining all fibers in the bundle. Regions of the cell periphery can be difficult to distinguish from the background, because the fluorescence intensity gradually diminishes at the cell perimeter. In order to prevent background interference from fibers underneath the cell periphery regions, the fibers with the top 30 peak intensities were chosen for the final analysis. The threshold was set at 75% for each fiber, and the residence time was considered to be the time above that threshold. The average residence times calculated for this series of experiments are shown in FIG. 3C. The average residence time for the control fiber was 7.5±7.4 frames (10 seconds per frame). Exposure to 200 nM nocodazole resulted in a residence time of 13.2±8.0 frames, and exposure to 300 nM nocodazole resulted in 19.0±9.4 frames.

The whole-cell and subcellular imaging experiments were each performed multiple times to ensure repeatability. In each series of experiments an increase in nocodazole concentration corresponded to an increase in residence time.

Cell Culture

NIH/3T3 fibroblasts cells were cultured using standard protocols 35 and subcultured twice per week to prevent confluency. Cell growth medium (DMEM+) contained Dulbecco's Modified Eagle Medium, 50 units/mL penicillin, 50 mg/mL streptomycin, 2 mM glutamine, 10% calf serum, and 1.85 g/L sodium bicarbonate. The cells were plated on untreated 100 mm petri dishes, and incubated at 37° C. in 5% carbon dioxide.

Cells were allowed to grow to near-confluency during each subculture cycle. Cells were then removed from the dish surface by adding trypsin, to cleave specific amino acid sequences. The cell suspension was then diluted with DMEM+, which includes serum to deactivate the trypsin. Cell were centrifuged and resuspended with an appropriate amount of medium, then allowed to settle and adhere to petri dishes for further growth.

Fiber Preparation

Fiber bundle specifications are detailed in Table 1. All fibers were polished using successive aluminum oxide lapping films (30, 12, 9, 3, 1, 0.3 mm), then rinsed with deionized water before further treatment.

TABLE 1
Fiber specifications.

| | | | Fiber |
|---|---|---|---|
| Manufacturer | Outer Diameter (mm) | Core Diameter (mm) | Count |
| Schott | 1 | 25 | ˜6000 |
| Illumina | 1 | 4.5 | ˜24,000 |

*Outer diameter is the diameter of the fiber bundle, and core diameter refers to the size of each individual fiber in a bundle

Prior to cell dispersal over the fibers, fibronectin was adsorbed to all fibers by exposure to 50 mg/mL fibronectin at 4° C. overnight. As a major constituent of the extracellular matrix, a layer of fibronectin allows the cells to adhere and migrate more easily than untreated glass or plastic.

Cell Labelling

Adherent cells were brought into suspension by exposing the plate to 0.05% trypsin-EDTA. The suspension was washed with DMEM+ to remove excess trypsin, then centrifuged at 1500 RPM for 10 minutes and resuspended in 2 mL serum-free DMEM. A 10 mL aliquot of Vybrant DiO (DiOC; excitation: 490 nm; emission: 502 nm) cell labeling solution was added to the cell suspension, and mixed by pipeting to form a 5 mM dye solution. The suspension was incubated for 15 minutes to allow the dye to associate with cell membrane. DMEM+ was then added to the suspension to a final volume of 20 mL. The suspension was washed twice with 20 mL of DMEM+ to remove excess dye from the solution. Cells were resuspended in an appropriate amount of culture medium, depending on suspension density. After labeling, the cell suspension was pipetted onto the fiber bundle, and allowed to settle and adhere. These cells sink to the surface within 15 minutes due to their relatively large size (25 micron diameter in suspension), and attach to the surface in less than 2 hours, after which they begin to migrate (data not shown).

Instrumentation

Fluorescence measurements were obtained through an IX81 Olympus Inverted Microscope. Excitation light is filtered and passed through the fiber bundle, interacting with membrane-bound fluorescent molecules near the distal face of the fiber (FIG. 1). The emission signal is passed back through the fiber, a microscope objective, and emission filter and then detected by a Cooke Sensicam CCD camera (The Cooke Corporation, Auburn Hills, Mich.) for whole-cell migration, and a Hamamatsu Orca CCD Camera (Hamamatsu City, Japan) for subcellular imaging. Data were collected using the IPLab image acquisition program (Scanalytics Inc, Fairfax, Va.).

Recording of Cell Migration

Timelapse recording was performed to determine the extent of cell migration or membrane motion across the fiber surface. Prior to recording, the fiber was washed twice with carbonate-free DMEM+. For drug-exposure, nocodazole was diluted in carbonate-free DMEM+. Cell movement was recorded on delta T4 dishes (data not shown), as well as on Schott and Illumina fiber bundles. The delta T dish culture system was used for its ability to maintain the temperature at 37° C. All movement was recorded using a fluorescein filter set. Images were recorded using a 5× objective for low-resolution fibers, and a 10× objective for high-resolution fibers. The total experimental time, exposure time, and time in between frames are detailed in Table 2.

TABLE 2
Summary of experimental times

| | Total Time | | Time in between |
|---|---|---|---|
| Manufacturer | (min) | Exposure Time (s) | Frames (s) |
| Schott | 3600 | 5 | 600 |
| Illumina | 5 | 1 | 9 |

Cell Viability

After the elapsed experimental period, cells were determined to be alive via a “dead” assay consisting of 4 mM ethidium homodimer, prepared in carbonate-free DMEM+. The dead assay was taken from a Live/Dead Viability/Cytotoxicity kit from Molecular Probes. DiO and EthD show virtually no overlap in excitation/emission wavelengths, being 484/501 nm and 528/617 nm, respectively. Cells were determined to be alive after early experiments, even after nocodazole exposure.

Discussion

Accordingly, we have demonstrated a fiber-optic based platform for monitoring cell migration that provides a simple whole cell migration assay and significantly reduces experimental time through the use of subcellular imaging. For whole-cell imaging, the increase in residence time seen upon the addition of nocodazole is due to the restricted movement of the cell. For subcellular imaging, the increase in residence time is most likely due to a decrease in intracellular traffic, caused by the lack of formation of a dynamic cytoskeleton. Microtubules have been shown to be responsible for intracellular transport of organelles, acting as a track for the motor proteins kinesin and dynein. Without a dynamic cytoskeleton, these tracks cannot be formed and organelles cannot be brought to their intended destinations. It has been shown that the saltatory movements of a variety of structures decreases upon the addition of nocodazole. Several membranous structures have been shown to have extensive interactions with the microtubule framework including the endoplasmic reticulum, mitochondria, lysosomes, and endosomes. The incorporation of dye into these membranous structures allowed their movement and arrest to be seen through an optical fiber. The technique in its current form is able to screen antimigratory compounds that target the cytoskeleton in cells that require an intact microtubule array to both migrate and perform intracellular transport. The method could be easily modified to detect changes in other cell types and structures, by altering the drugs and dyes used for experimentation.

Using an optical fiber platform significantly decreases experimental time from a minimum of several hours to 5 minutes. Examination of subcellular features of individual cells that correlate with cell migration proved to be much faster than examining migration itself. This decrease in experimental time gives the fiber-optic platform a significant advantage over more common techniques, which typically take hours to perform a single experiment, and should enable high throughput cell migration assays. In addition, several bundles can be imaged simultaneously using the same imaging system, thereby further increasing throughput.

The technique evolved to enable us to run simultaneous experiments by packing multiple fiber bundles together. In preliminary experiments, longer, more flexible fibers were used in an attempt to image multiple fibers at the same time. The proximal ends of several fiber bundles were fused and polished to form a planar surface for focusing. The free distal ends of each fiber bundle were inserted into separate wells of a 96 well microplate. The fused end brings all the fiber bundles together so that several bundles can be monitored simultaneously, while exposing them to different conditions on their distal ends. A suspension of dyed cells was pipetted into each well of the plate and then imaged after the cells had adhered to each of the fiber bundles. This technique can potentially compress experimental time even further, by running several fiber-based experiments simultaneously. The potential capacity of this type of experiment may only be limited by the number of fibers that can be fit into the microscope's field of view or by the area of the CCD chip. This parallel approach may enable scores of rapid, subcellular imaging experiments to be performed concurrently. In addition to their use in running parallel experiments, the use of long, flexible fibers demonstrates remote sensing, as hundreds of centimeters can separate the assay and imaging locations as required. In another embodiment, the present invention utilizes a planar substrate with one or more fiber bundles embedded in the substrate.

Another important advantage of this technology is the potential to reduce the number of cells and amount of drug used for each migration assay. The current experimental setup is based on using delta T4 dishes, but other systems may use a vessel that more closely matches the fiber diameter, minimizing the amount of drug and cells used in an experiment. This reduction in size may allow the use of a cell line that is difficult to grow and maintain, including primary cells. Individual fiber sizes can be further decreased, augmenting the range of applications for subcellular imaging. While the examples describe assays that are specific for cytoskeleton-affecting compounds, the platform should allow for a wide range of applications with the use of different dyes and fiber sizes. These applications may include monitoring particle movement, multi-organelle interactions, and cell-substrate events.

What is claimed is:

1. A method of detecting cell migration, the method comprising:
providing a plurality of cells on a substrate comprising a plurality of detection units, wherein each of said cells is disposed over at least one of said detection units; and
detecting cellular movement of at least one of said cells.

2. The method of claim 1, wherein each of said cells is disposed over more than one of said detection units.

3. The method of claim 1, further comprising adding an analyte of interest prior to said detecting cellular movement.

4. The method of claim 1, further comprising:
adding an analyte of interest after said detecting cellular movement; and
detecting cellular movement of at least one of said cells a second time after adding said analyte.

5. The method of claim 4, further comprising comparing data from said detecting cellular movement steps to identify any effect of said analyte of interest on cellular movement.

6. The method of claim 5, further comprising comparing data from both of said detecting cellular movement steps to measure any effect of said analyte of interest on cellular movement.

7. The method of claim 1, wherein said detecting cellular movement comprises detecting movement of at least one of said cells in relation to at least one of said detection units.

8. The method of claim 1, wherein said detecting cellular movement comprises detecting movement of a subcellular component of at least one of said cells in relation to at least one of said detection units.

9. The method of claim 1, wherein said detecting cellular movement comprises detecting residence time in relation to at least one of said detection units.

10. The method of claim 9, wherein said residence time is an amount of time during which at least a portion of at least one of said cells is disposed above at least one of said detection units.

11. The method of claim 1, wherein at least one of said plurality of cells comprises a label.

12. The method of claim 11, wherein the label is a fluorescent label.

13. The method of claim 11, wherein a subcellular component of said at least one of said plurality of cells comprises the label.

14. The method of claim 11, wherein said detecting cellular movement comprises detecting movement of at least one of said labels in relation to at least one of said detection units.

15. The method of claim 11, wherein said detecting cellular movement comprises detecting residence time of at least one of said labels in relation to at least one of said detection units.

16. The method of claim 15, wherein said residence time is an amount of time during which at least a portion of at least one of said labels is disposed above at least one of said detection units.

17. The method of claim 1, wherein said providing a plurality of cells comprises providing at least two cell types on said substrate.

18. The method of claim 1, wherein each of said plurality of cells has a diameter greater than a diameter of any of said plurality of detection units.

19. The method of claim 1, further comprising testing viability of said cells.

20. A composition for detection of cellular migration, the composition comprising:
a substrate comprising a plurality of detection units; and
a plurality of cells distributed on said substrate such that each of said cells is disposed over at least one of said detection units.

21. The composition of claim 20, wherein each of said cells is disposed over more than one of said detection units.

22. The composition of claim 20, further comprising a detection component.

23. The composition of claim 22, wherein the detection component comprises a microscope.

24. The composition of claim 20, wherein said substrate comprises a cell adhesion facilitator.

25. The composition of claim 22, wherein the composition is a biosensor array.

26. The composition of claim 20, wherein said substrate is at least one fiber optic bundle.

27. The composition of claim 26, wherein said detection units are fiber strands.

28. The composition of claim 20, wherein each of said detection units is a discrete optically viewable site in said substrate.

29. The composition of claim 20, wherein said substrate is selected from the group consisting of plastic and glass.

30. The composition of claim 20, further comprising an analyte of interest distributed on said substrate.

31. The composition of claim 20, wherein at least one of said plurality of cells comprises a label.

32. The composition of claim 31, wherein the label is a fluorescent label.

33. The composition of claim 20, wherein said plurality of cells comprises at least two cell types.

34. The composition of claim 20, wherein each of said plurality of cells has a diameter greater than a diameter of any of said plurality of detection units.

35. The composition of claim 20, wherein said detection units have a density ranging from about _{—} to about _{—}

36. The composition of claim 20, where said detection units have a size ranging from about _{—} to about _.

37. The composition of claim 20, further comprising a temperature regulator.

38. The composition of claim 20, further comprising a computer.

39. The composition of claim 38, wherein said computer is configured to analyze a signal from said substrate.
